(19) United States
(12) Patent Application Publication
(10) Pub. No.: US 2021/0319873 A1
(43) Pub. Date: Oct. 14, 2021

(54) FITNESS PROGRESS TRACKING WITH MULTIVARIABLE INPUT

(71) Applicant: ChallengeRunner LLC, Cincinnati, OH (US)

(72) Inventor: Joseph Kennedy, Hamilton, OH (US)

(21) Appl. No.: 17/228,081

(22) Filed: Apr. 12, 2021

Related U.S. Application Data
(60) Provisional application No. 63/009,772, filed on Apr. 14, 2020.

(51) Int. Cl. G16H 20/30; G16H 20/60; G16H 50/70; G16H 40/67; G01C 22/00; A63F 13/65; A63F 13/46; A63F 13/52; A63F 13/533; G06F 3/0481; G06T 11/00

(52) U.S. Cl. CPC G16H 20/30; G16H 20/60; G16H 50/70; G16H 40/67; G01C 22/006; A63F 13/65; A63F 2300/69; A63F 13/52; A63F 13/533; G06F 3/04817; G06T 11/00; A63F 2300/308; A63F 2300/61; A63F 13/46

(57) ABSTRACT

A software platform for managing fitness challenges or other competitive group challenges may include a multivariable progress tracking feature and interface. Multivariable progress tracking may be configured by defining a plurality of tracked inputs such as steps taken, minutes spent exercising, hydration levels, and weight loss. A relationship may be configured between multivariable inputs and a point total goal for the challenge such that each type of input from the user will contribute a variable amount of points towards a total goal. Multivariable inputs may be manually provided by users, or may be automatically provided by a wearable device or other smart connected device. As user progress is tracked by point total, the system may provide users customized interface that allow input of the tracked inputs, and may display leaderboards and progress illustrations that show the progress of the entire group.

### PRIORITY

This application claims the priority of U.S. Provisional Patent 63/009,772, filed Apr. 14, 2020, and titled “Fitness Progress Tracking with Multivariable Input,” the entire disclosure of which is incorporated herein by reference.

### FIELD

The disclosed technology pertains to a system for tracking fitness progress with multiple input variables.

### BACKGROUND

Competitive fitness activities have grown in popularity as the importance of exercise and the risks of a sedentary lifestyle have become more apparent and more widely known. Timed running activities occur every week across the United States, if not every day, with thousands of competitive five kilometer or “5k” runs occurring every year. With the rise popularity of running and other activities amongst both the ultra-competitive and the casual competitor, various technologies have also been developed to help competitors track their progress in preparing for an event, and track their results at an event.

Competitive fitness activities have also become a popular group activity with groups of friends or employees often collectively participating in training and performance of the event. Some software platforms have provided ways for such groups to track their training and performance in an event, and in some cases have been provided to employees by a company in order to promote health, fitness, and teamwork.

Such interfaces and systems generally provide very basic controls and interfaces that allow users to view their own training progress, and in some cases view the training progress of others. However, such information is frequently provided in simple formats such as numerical or text progress reports that simply report a time spent jogging, a number of miles walked, or a number of steps taken. Many users find such basic information to provide little inspiration either for individual success, or group successes.

What is needed is an improved system for tracking group and individual progress towards a competitive goal.

### BRIEF DESCRIPTION OF THE DRAWINGS

The drawings and detailed description that follow are intended to be merely illustrative and are not intended to limit the scope of the invention as contemplated by the inventors.

FIG. 1 is a flowchart of an exemplary set of steps that could be performed with a system to configure and provide multivariable progress tracking;

FIG. 2 is a flowchart of an exemplary set of steps that could be performed with a system to receive progress information and display progress tracking interfaces;

FIG. 3 is a flowchart of an exemplary set of steps that could be performed with a system to provide user pairing;

FIG. 4 is a screenshot of an interface that may be used to configure multivariable progress tracking;

FIG. 5A is a screenshot of an exemplary interface that shows a progress leaderboard in a first state;

FIG. 5B is a screenshot of an exemplary interface that shows the progress leaderboard in a second state;

FIG. 6A is a screenshot of an exemplary interface that shows tracked progress as a land path; and

FIG. 6B is a screenshot of an exemplary interface that shows tracked progress as a space path.

### DETAILED DESCRIPTION

The inventor has conceived of novel technology that, for the purpose of illustration, is disclosed herein as applied in the context of fitness and competitive activity tracking. While the disclosed applications of the inventors' technology satisfy a long-felt but unmet need in the art of fitness and competitive activity tracking it should be understood that the inventors' technology is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

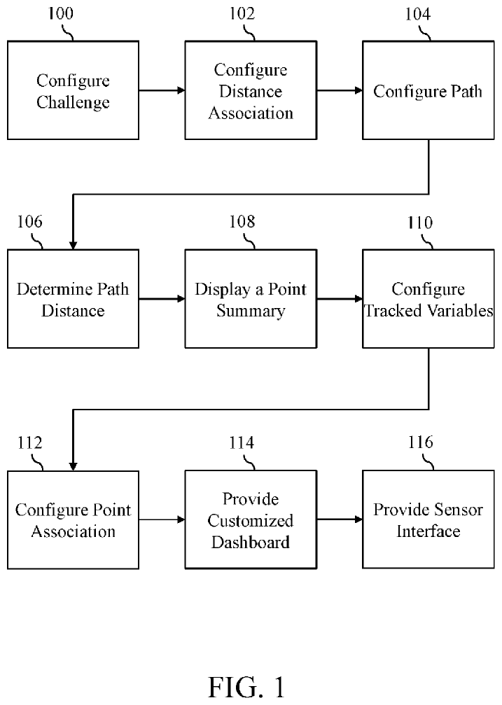

Turning now to the figures, FIG. 1 is a flowchart of an exemplary set of steps that could be performed with a system to configure and provide multivariable progress tracking. An appropriate system may include one or more physical, virtual, or cloud servers (e.g., each including processors, memory, communication devices, or other devices) configured to provide a web interface, webservice, or other software interface to users that access the system via a web browser, mobile software application, or other software interface. In some implementations, multivariable progress tracking may be included in a competitive group progress tracking application such as may be used to allow a group of individuals (e.g., a social group, a group of employees) to competitively track their progress towards a common goal such as training for a competitive event or living a healthier lifestyle. In such a system, configuration may first include configuring (100) a challenge, which may include identifying the users that will be part of the challenge, configuring the dates that the challenge will be available, and configuring the end goal of the challenge. The end goal of the challenge may be configured as a total number of points that must be achieved to complete the challenge.

A user may also configure (102) a distance association which may be used to relate point progress in the challenge to various real-world distances or locations, which may be used to provide visual illustrations that indicate progress. As an example, a challenge may be configured to illustrate progress in relation to a distance traveled around the earth. With a circumference of 24,901 miles, the challenge may be configured (100) with a goal of 24,901, and a distance association (102) of 1 point equal to 1 mile (e.g., a user reaching 24,901 points will have traveled around the earth). Progress tracking may then be illustrated to users participating in the challenge as a visual illustration of the earth with indicators showing where each competitor is located based upon their points.

Progress tracking may be configured in other ways beyond a distance around the earth, and a user may configure (104) any desired path for the progress tracking. This may include configuring a particular route relative to the earth, within the solar system, or within another spatial context. In some implementations, path configuration (104) may include selecting two or more locations from a list or searchable collection of real-world locations, such as selecting Las Vegas, Nev. as a starting point and Sacramento, Calif. as an endpoint. Another example may include selecting the Earth as a starting point, and another planet or location within space as an endpoint. Other examples may include configuring starting points, midpoints or milestones, and endpoints based upon fictional locations (e.g., such as locations in a sci-fi setting) or other abstract locations.

With one or more points of a path configured (104), the system may determine (106) the distance of the path, both between individual locations and in total. Distance between locations may be determined as a straight-line distance or may be measured along roadways, interstates, or other methods of travel between locations. Using a prior example, a configured path (104) that circled the globe would be associated with a determined (106) path distance of 24,901 miles. The path distance and distance association may then be used to display (108) a point summary for the path that indicates the total number of points needed to reach each location.

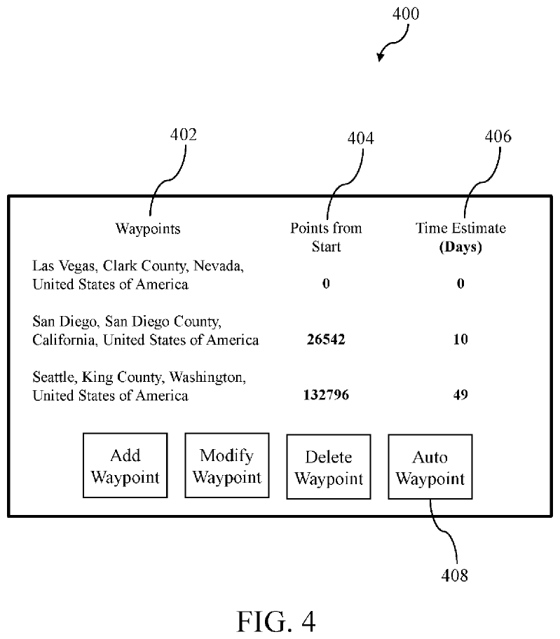

FIG. 4 shows an example of an interface that may be displayed (108) to show a point summary for the path. The interface (400) may be displayed to indicate to a user the locations of the configured path and the number of points required to reach each location in order to aid the user in configuring a progress tracker that will be challenging for a group but not impossible. A number of locations, milestones, or waypoints (402) may be displayed, and a total number of points (404) to reach each in sequence may also be displayed. In some implementations, the system may also determine an estimated time (406) to reach each location, with such estimates being based upon static variables or based upon average times to attach such point scores using historical data from past challenges available to the system. Time estimates may also take into account point sources of the multiple input variables that contribute to each users point total, as will be described in more detail below.

The interface (400) may also include a number of user controls to add additional locations to the path, modify locations, delete locations, or automatically suggest locations (408). Automatic location suggestion may receive input such as a desired duration for the challenge (e.g., 2 months) and then may automatically suggest two or more locations to produce a path and point total that is likely to be completed in the approximate time frame of the desired duration. As an example, with reference to FIG. 4, a user may select to automatically populate waypoints (408) and provide a desired duration of 7 weeks. The system may examine historical progress tracking data and determine that a route from Las Vegas, to San Diego, and then to Seattle with the configured point total and tracked variables is estimated to be completed in 49 days, and may automatically populate the path.

With reference to FIG. 1, the system may also allow a user to configure (110) a plurality of tracked variables and configure (112) a point association for each tracked variable. The multiple variables tracked by the system may be selected from a variety of inputs and may include inputs related to fitness activities, nutritional choices, ecologically friendly activities (e.g., use of public transportation or bikes), lifestyle choices, or other activities. Fitness related inputs that the system may be configured to receive may include steps taken, miles traveled on foot, stairs climbed, heart rate elevation, distance traveled on a bike, exercises performed at a gym, body fat percentage, weight loss, or other fitness related activities that contribute to the overall goal of the challenge. Nutritional choices that the system may be configured to receive may include calories consumed, glasses of water consumed, avoidance of certain foods, consumption of certain foods, or other dietary choices that contribute to the overall goal of the challenge. Lifestyle related inputs may include time spent in a gym, time spent in a park, time spent in positive social settings, or other lifestyle choices that may contribute to the overall goal of the challenge. Tracked (110) variables may be configured for manual input by users (e.g., specifying a number of glasses of water consumed or steps taken during a day), automatic input (e.g., number of steps taken during a day provided by a step tracing device, weight loss provided by a wirelessly connected scale), or both.

A configured (112) point association creates a relationship between the total points and distance for the challenge and each discrete input type. Point associations may be variable configured to provide greater weight for certain inputs, as may be desirable for a particular challenge type. As an example of point weighting, for a challenge that is desired to promote healthy living with a primary goal of weight loss, inputs such as minutes spent exercising (e.g., 10 points per minute), adequate hydration (e.g., 10 points per glass), and measurable weight loss (e.g., 100 points per ounce) may each by associated with a high number of points as compared to secondary goals, such as ecologically friendly activity (e.g., 5 points for using public transit).

Based upon the configured (110) variables, the system may then provide (114) a customized dashboard and sensor interfaces (116) to the users participating in the challenge. The customized dashboard may be presented to competing users that access the system and will specify the types of input that the users may provide, and provide an interface for manually inputting such information (e.g., specifying a number of glasses of water consumed each day) or verifying automatically provided information (e.g., confirming a number of steps taken that are provided by a wearable step tracker). Providing (116) the sensor interface may include providing a software interface that each user's mobile device, wearable devices, or other connected devices may communicate with to automatically provide inputs (e.g., devices may communicate directly with the system via the software interface, or may communicate with the user's smartphone or other gateway device that itself communicates with the system via the software interface).

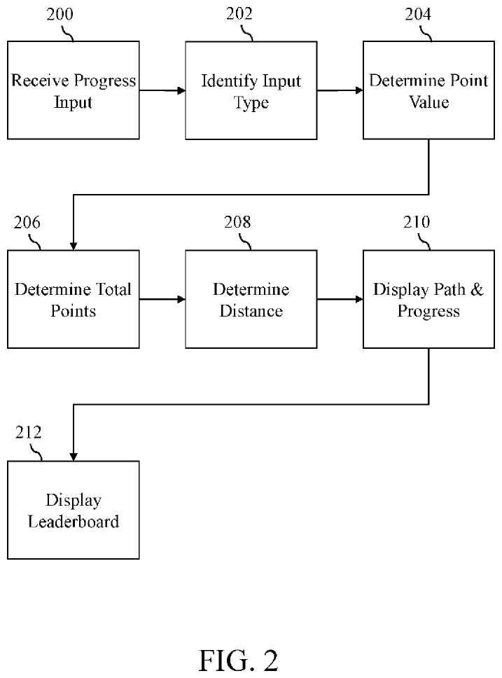

FIG. 2 is a flowchart of an exemplary set of steps that could be performed with a system to receive progress information and display progress tracking interfaces. Multiple progress inputs (e.g., steps, miles run, glasses of water consumed, etc.) may be received by the provided dashboard (114) and/or sensor interfaces (116). As inputs are received (200) they are identified (202) to determine their type (e.g., does a numerical input indicate a number of steps or a number of minutes spent exercising?) and a point value is determined (204) for each input based upon the configured (112) point associations for each tracked input type. As individual inputs are received and their point values determined, a total number of points may be determined (206) for each user that is participating in the challenge. Each user's total points may then be used to determine (208) the equivalent distance for purposes of progress illustration based upon the configured (102) distance association, as has been described.

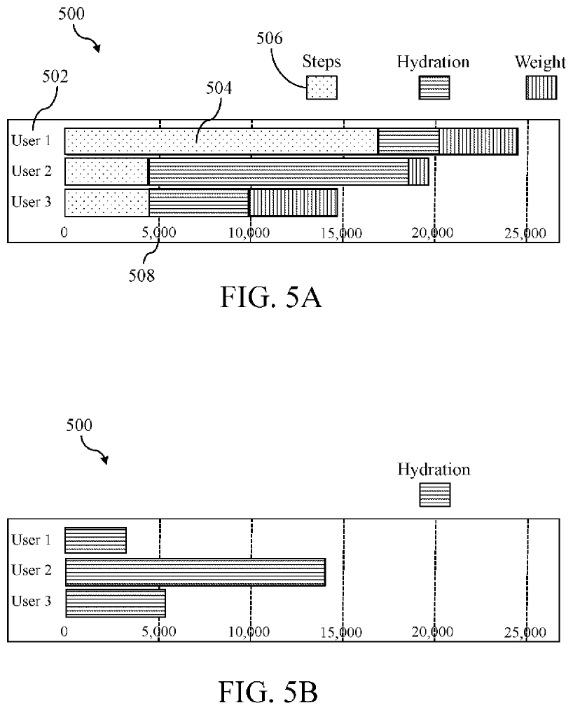

The system may then display (210) an interface showing the configured path and user progress for one or more users, and may also display (212) a leaderboard showing progress for a plurality of users. FIGS. 5A and 5B show examples of an interface that may be displayed (212) when showing a leaderboard interface. In FIG. 5A, an interface (500) shows a plurality of users (502) that may be identified based upon a username, user identifier, photograph, avatar, or other identifying information. Each user may also have a visual indicator of their point total (504) in the form of a graph, chart, or other shape that conveys a relative point total or point progress compared to other users. In the interface (500) that is shown, each users point total (504) is shown as a bar whose length is related to a point scale (508) along the bottom of the chart (e.g., User 3 has nearly 15,000 total points, while User 1 has nearly 25,000 points). The point totals (504) may also provide additional details related to the multivariable composition of the point totals. As an example, a legend (506) shows visually distinctive elements (e.g., patterns, colors, other visual effects) that compose the point total (504) bar, and indicate the source of points for that user. For example, the point total for User 1 is shown to be composed of about 17,000 points from daily steps, about 3,000 points from daily hydration, and about 4,000 points from weight loss. The interface (500) is shown in FIG. 5B in a second state, where the leaderboard view has been filtered to only show hydration point totals. As can be seen, when focusing only on hydration User 2 leads with nearly 14,000 points from hydration, while User 1 has around 3,000 points from hydration. The filtered view of FIG. 5B may be displayed when a user selects or interacts with an interface element in the interface (500), which may include a drop-down menu, checkbox, radio button, or other clickable element (e.g., by clicking on an element of the legend (506)).

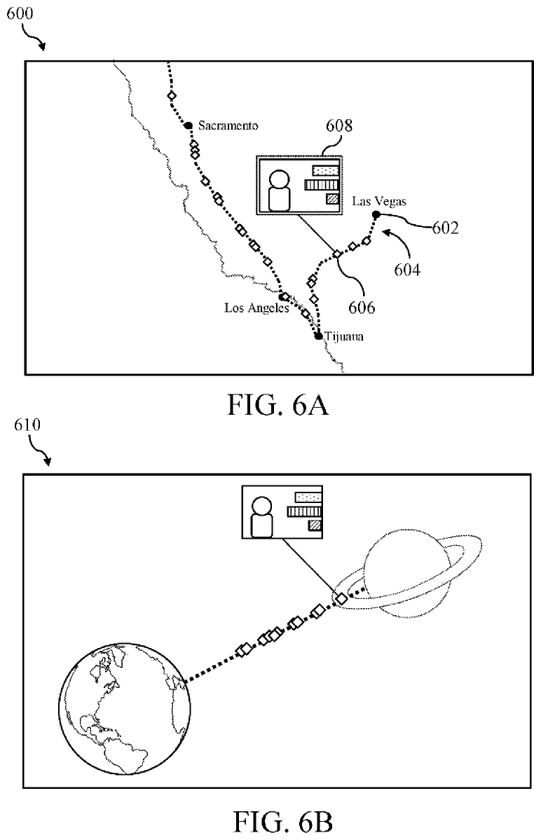

FIGS. 6A and 6B each show examples of a displayed (210) path. The displayed (210) path may be shown in the same or a different interface as the displayed (212) leaderboard or other challenge and progress related information. FIG. 6A is a screenshot of an exemplary interface (600) that shows tracked progress as a land path through a portion of the western United States. An origin point (602) is shown and marked as “Las Vegas, and a dotted line (604) shows a path from Las Vegas to a number of other subsequent locations (e.g., Tijuana, Los Angeles, and Sacramento). A plurality of user markers (606) may be shown along the path, with their position on the path being determined by that users point total and the configured (102) association between distance and points. Portions of the interface (600) such as the user markers (606) may be interacted with (e.g., clicked on, hovered over) to show additional information such as a user card (608) that shows a user's point totals from various inputs (e.g., a bar showing point totals from steps, hydration, weight lost, etc.) and may also show a user's name, photograph, avatar, or other identifying information.

FIG. 6B is a screenshot of an exemplary interface (610) that shows tracked progress as a space path, and that may function similarly to the interface (600) shown in FIG. 6A. In the interface (610) the Earth is shown as an origin point, with Saturn being a destination. A dotted path line connects to the two locations, and a number of interactive user markers are shown along the path line based upon their point totals and distance association. Other path illustrations that may be displayed (210) may be based upon fictional locations (e.g., locations from a sci-fi or fantasy setting) or locations that are somewhat abstracted so that they relate to humans (e.g., such as a path that an electrical synapse travels along the nervous system, or an electrical signal traveling through a microprocessor). Display of progress may be further abstracted beyond locations, and may instead indicate progress through other visual illustrations besides spatial progress. This may include visual progress such as an avatar that becomes more muscular or more athletic looking as point progress increases, a house that becomes larger and more lavishly furnished based on point progress, an avatar that levels up or becomes more powerful based on point progress, or other forms of visual gamification that reward a user's point progress.

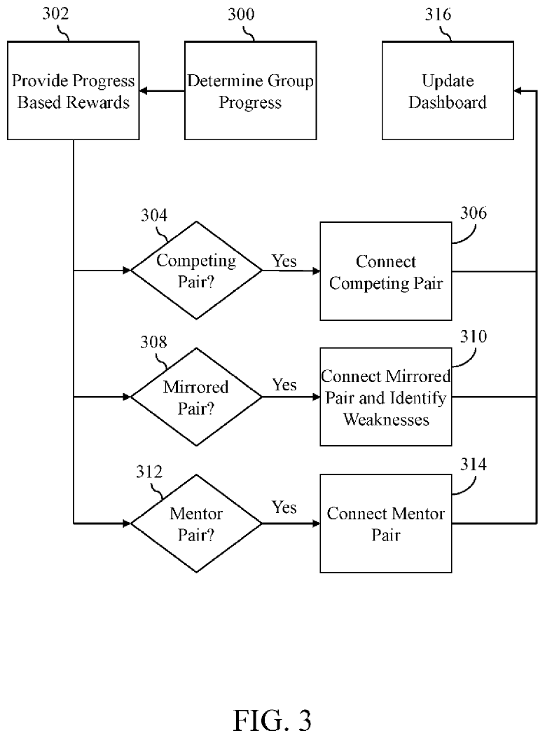

In addition to providing leaderboard and path display to promote competition and progress, the system may also perform targeted pairing and connection of users participating in the competition based upon their multivariable inputs. As an example, FIG. 3 is a flowchart of an exemplary set of steps that could be performed with a system to provide user pairing. When group progress is determined (300), which may occur in real-time as user inputs are received and leaderboards are updated based on total points, or may occur daily or weekly based on a scheduled update, the system may provide (302) one or more progress based rewards to users. This could include digital rewards for reaching certain locations (e.g., a digital trophy that is displayed with your avatar for reaching Tijuana from Las Vegas), digital rewards for being in the top three of point totals for a week, or other digital or physical rewards based upon various inputs and/or point totals.

The system may also analyze each users point totals and the composition of their point totals to determine if any targeted user pairings might benefit one or more of the competitors. For example, the system may identify a competing pair (304) of users that are performing very similarly to each other in point totals or individual input point totals, and may connect (306) that competing pair. Connecting (306) the competing pair may include creating a visual association between the users in the dashboard and/or displayed path (e.g., the user markers (606) on the path may be connected by a line to show that the users are working together and supporting each other), may include updating the dashboard and leaderboard for each of the paired users to provide them communication options for directly messaging each other, may provide targeted notifications to each of the pair when their point totals change, or may provide text messages, emails, or other electronic communications that recommend the users connect with other and support each other through the competition. These types of automatic connections may allow users to connect with and support each other through competition with a minimal amount of effort. As an example, a generalized messaging system may not be as useful to users since they may receive messages from too many other users or may avoid initially contacting other users. An automated targeted pairing of two competing users that provides a communication channel only usable by those two users is perceived differently than a generalized messaging system, and so may create a more personal connection for some users.

The system may also analyze the user progress and identify a mirrored (308) pairing of users based upon their point totals. A mirrored (308) pair of users includes a user that is performing well in a first group of inputs (e.g., steps, minutes exercising) but poorly in a second group of inputs (e.g., hydration, weight loss), and a second user whose performance is the opposite across inputs. As an example, a first user may be reporting high numbers of steps and a high level of exercise and fitness activity, but may be reporting very low weight loss numbers despite high activity. A second user may be reporting low numbers for steps and exercise, but may be reporting high weight loss. The system may connect (310) the mirrored pair as described above (e.g., personalized messaging, leaderboard and path illustration linking), and may identify the weaknesses and strengths of each user to the other. Users may find this personalized connection and communication channel useful to share tips and support with each other. In the example above, the first user may inspire the second user to increase their exercise and achieve even greater results, while the second user may share tips on food and hydration that allow the first user to improve their weight loss results.

The system may also analyze user progress and identify a mentor (308) pairing of users based upon their point totals. A mentor (308) pair may include identifying a first user that is performing well based upon their progress totals, and a second user that is not performing well. As an example, the first user may be leading the challenge, or may be performing within a range of the average, while the second user may be minimally participating (e.g., very low point totals, or inconsistent input of data that might indicate a lack of interest in participating). The system may connect (314) an identified mentoring pair, and may provide personalized messaging channels, visual indicators, or other connections. Users may find such a personalized connection and communication channel useful as compared to a more generalized connection or messaging channel, and may promote the first user to share support and advice with the second user, or may promote the second user to reach out for support from the first user. Other types of user pairing exist, and such variations and additional examples will be apparent to those of ordinary skill in the art in light of this disclosure.

It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A system for tracking fitness progress comprising a server that includes a processor, the processor configured to:
(a) create a plurality of fitness challenges based upon user input, wherein each of the plurality of fitness challenges comprises a time period, a virtual route, a point system that corresponds to progress along the virtual route, and a set of one or more input variable types, wherein each of the set of input variable types comprises a point value that is associated with the point system;
(b) receive inputs from a plurality of user devices that are associated with a plurality of participants in a fitness challenge of the plurality of fitness challenges, during the time period for the fitness challenge, wherein the inputs correspond to one of the set of two more input variable types;
(c) determine a score for each of the plurality of participants based on inputs received from that participant and the point values associated with the one or more input variable types, and determine a position for each of the plurality of participants on the virtual route based upon the score for that participant; and
(d) provide a dashboard to be displayed on the plurality of user devices, wherein the dashboard comprises:
(i) one or more input controls that correspond to the one or more input variable types, wherein the one or more input controls are each usable to provide input with a pre-determined input variable type to the server;
(ii) a visual depiction of a location through which the virtual route travels and a visual depiction of the virtual route overlaid upon the visual depiction of the location; and
(iii) a plurality of visual icons that correspond to each of the plurality of participants overlaid upon the visual depiction of the virtual route based on the determined position for that participant.

2. The system of claim 1, wherein the set of one or more input variable types comprises:
(a) a number of steps taken by a participant during a day;
(b) a volume of water consumed by the participant during a day; and
(c) an amount of weight lost by the participant.

3. The system of claim 2, wherein the set of one or more input variable types further comprises:
(a) a number of calories consumed by the participant during a day;
(b) an amount of time spent at a gym; and
(c) an indication of the occurrence of an ecologically friendly activity.

4. The system of claim 2, wherein each of the set of one or more input variable types is associated with a different point value in miles or kilometers that corresponds to progress along the virtual route.

5. The system of claim 1, wherein the set of one or more input variables types includes a number of steps taken by a participant during a day and a manual input variable, wherein the processor is configured to:
(a) receive the manual input variable via a manual input to the dashboard; and
(b) receive the number of steps taken from a step count sensor of a user device.

6. The system of claim 1, wherein:
(a) the processor is further configured to store a configured digital reward criteria, and determine, based upon the determined position for each of the plurality of participants, a subset of participants that have satisfied the digital reward criteria; and
(b) the dashboard further comprises, for any of the plurality of visual icons that are associated with the subset of participants, a visual indication of the digital reward.

7. The system of claim 1, wherein the processor is further configured to:
(a) identify two or more participants of the plurality of participants that have a similar point total for at least one of the one or more input variable types; and
(b) for each of the two or more participants, cause the dashboard to further comprise a visual indication of the other participant that they are competing with.

8. The system of claim 1, wherein the processor is further configured to:
(a) identify two or more participants of the plurality of participants that have mirrored point total for at least two of the one or more input variable types; and
(b) for each of the two or more participants, cause the dashboard to further comprise a visual indication of the other participant that they are mirrored with, and an input control usable to exchange electronic communications with the other participant.

9. The system of claim 1, wherein the processor is further configured to:
(a) identify a high performing participant and a low performing participant of the plurality of participants based on the determined score for each; and
(b) for the high performing participant and the low performing participant, cause the dashboard to further comprise a visual indication of the other, and an input control usable to exchange electronic communications with the other.

10. The system of claim 1, wherein the processor is further configured to provide an administrative dashboard that is usable to provide inputs for creation of fitness challenges, wherein the administrative dashboard comprises a set of administrative input controls usable by an administrator to specify a desired duration for the fitness challenge, the set of one or more input variable types, and the point value for each of the one or more input variable types, wherein the processor is configured to:
(a) determine a suggested virtual route based on the desired duration, the set of two more input variable types, the point value for each of the one or more input variable types, and a set of historic fitness challenge information that describes the results of past fitness challenges;
(b) provide a description of the suggested virtual route via the administrative dashboard; and
(c) in response to receiving input from the administrator, use the suggested virtual route as the virtual route.

11. The system of claim 1, wherein:
(a) the visual depiction of the location comprises a geographical map of a location on earth; and
(b) the visual depiction of the virtual route comprises a line that follows one or more roadways present at the location.

12. The system of claim 1, wherein the dashboard further comprises a set of interactive controls associated with the plurality of visual icons, wherein the set of interactive controls are configured to:
(a) detect an interaction with a visual icon of the plurality of visual icons; and
(b) display a user card describing a participant that corresponds to the visual icon, wherein the user card comprises:
(i) a name associated with the participant;
(ii) point totals for each of the two more input variable types associated with the participant; and
(iii) an image associated with the participant.

13. The system of claim 1, wherein the processor includes two or more processors that are in communication with each other directly or over a network.

14. A method for tracking fitness progress comprising using a server that includes a processor, the method comprising, with the processor:
(a) creating a plurality of fitness challenges based upon user input, wherein each of the plurality of fitness challenges comprises a time period, a virtual route, a point system that corresponds to progress along the virtual route, and a set of one or more input variable types, wherein each of the set of input variable types comprises a point value that is associated with the point system;
(b) receiving inputs from a plurality of user devices that are associated with a plurality of participants in a fitness challenge of the plurality of fitness challenges, during the time period for the fitness challenge, wherein the inputs correspond to one of the set of two more input variable types;
(c) determining a score for each of the plurality of participants based on inputs received from that participant and the point values associated with the two more input variable types, and determine a position for each of the plurality of participants on the virtual route based upon the score for that participant; and
(d) providing a dashboard to be displayed on the plurality of user devices, wherein the dashboard comprises:
(i) one or more input controls that correspond to the one or more input variable types, wherein the one or more input controls are each usable to provide input with a pre-determined input variable type to the server;
(ii) a visual depiction of a location through which the virtual route travels and a visual depiction of the virtual route overlaid upon the visual depiction of the location; and
(iii) a plurality of visual icons that correspond to each of the plurality of participants overlaid upon the visual depiction of the virtual route based on the determined position for that participant.

15. The method of claim 14, wherein the set of one or more input variable types comprises:
(a) a number of steps taken by a participant during a day;
(b) a volume of water consumed by the participant during a day; and
(c) an amount of weight lost by the participant.

16. The method of claim 14, wherein the set of one or more input variables types includes a number of steps taken by a participant during a day and a manual input variable, the method further comprising, with the processor:
(a) receiving the manual input variable via a manual input to the dashboard; and
(b) receiving the number of steps taken from a step count sensor of a user device.

17. The method of claim 14, further comprising, with the processor:
(a) identifying two or more participants of the plurality of participants that have a similar point total for at least one of the one or more input variable types; and
(b) for each of the two or more participants, causing the dashboard to further comprise a visual indication of the other participant that they are competing with.

18. The method of claim 14, the method further comprising providing an administrative dashboard that is usable to provide inputs for creation of fitness challenges, wherein the administrative dashboard comprises a set of administrative input controls usable by an administrator to specify a desired duration for the fitness challenge, the set of one or more input variable types, and the point value for each of the one or more input variable types, the method further comprising, with the processor:
(a) determining a suggested virtual route based on the desired duration, the set of two more input variable types, the point value for each of the one or more input variable types, and a set of historic fitness challenge information that describes the results of past fitness challenges;
(b) providing a description of the suggested virtual route via the administrative dashboard; and
(c) in response to receiving input from the administrator, using the suggested virtual route as the virtual route.

19. The method of claim 14, wherein:
(a) the visual depiction of the location comprises a geographical map of a location on earth; and
(b) the visual depiction of the virtual route comprises a line that follows one or more roadways present at the location.

20. A system for tracking fitness progress comprising a server that includes a processor, the processor configured to:
(a) create a plurality of fitness challenges based upon user input, wherein each of the plurality of fitness challenges comprises a time period, a virtual route, a point system that corresponds to progress along the virtual route, and a set of one or more input variable types, wherein each of the set of input variable types comprises a point value that is associated with the point system, and wherein the set of one or more input variables types comprises:
(i) a number of steps taken by a participant during a day;
(ii) a volume of water consumed by the participant during a day; and
(iii) an amount of weight lost by the participant;
(b) receive inputs from a plurality of user devices that are associated with a plurality of participants in a fitness challenge of the plurality of fitness challenges, during the time period for the fitness challenge, wherein the inputs correspond to one of the set of two more input variable types, wherein:
(i) the number of steps taken are received from a step count sensor of a user device; and
(ii) the volume of water consumed and the amount of weight lost are received from a manual input;
(c) determine a score for each of the plurality of participants based on inputs received from that participant and the point values associated with the two more input variable types, and determine a position for each of the plurality of participants on the virtual route based upon the score for that participant; and
(d) provide a dashboard to be displayed on the plurality of user devices, wherein the dashboard comprises:
(i) one or more input controls that correspond to the one or more input variable types, wherein the one or more input controls are each usable to provide input with a pre-determined input variable type to the server;
(ii) a visual depiction of a location through which the virtual route travels and a visual depiction of the virtual route overlaid upon the visual depiction of the location,
(A) wherein the visual depiction of the location comprises a geographical map of a location on earth; and
(B) the visual depiction of the virtual route comprises a line that follows one or more roadways present at the location; and
(iii) a plurality of visual icons that correspond to each of the plurality of participants overlaid upon the visual depiction of the virtual route based on the determined position for that participant.
